(19) United States
(12) Patent Application Publication
(10) Pub. No.: US 2016/0017373 A1
(43) Pub. Date: Jan. 21, 2016

(54) Bulk Availability and Production of Bioremediation Products

(71) Applicant: Satyanarayana Ganti, Dover, PA (US)

(72) Inventor: Satyanarayana Ganti, Dover, PA (US)

(21) Appl. No.: 14/164,336

(22) Filed: Jan. 27, 2014

Related U.S. Application Data
(60) Provisional application No. 61/788,348, filed on Mar. 15, 2013.

(51) Int. Cl. C12P 1/04

(52) U.S. Cl. CPC C12P 1/04

(57) ABSTRACT

A method for producing and storing a bioremediation product that includes active bacteria for bioremediation of hydrocarbon oil pollution includes storing the bioremediation product in bulk in ready-to-use form for later application at a pollution site. The bioremediation product has a typical shelf life of 2 years or more.

### FIELD OF THE DISCLOSURE

This disclosure relates to bulk availability and production of bioremediation products for spill response preparedness for open water and terrestrial oil spills.

### BACKGROUND OF THE DISCLOSURE

Bioremediation is a sustainable waste management technique that involves the use of microorganisms to remove or remediate pollutants. The microorganisms are seeded to physically access the pollutants and, if necessary, nutritional supplements are added to further support growth of the microorganisms sufficient to remove or remediate the targeted pollutants.

Hydrocarbons are the principle constituents of hydrocarbon oils such as crude oil and related byproducts or distillates: coal tar, diesel oil, gasoline, kerosene and the like. Hydrocarbon oils are composed of straight or branched saturated aliphatic, polycyclic aromatic hydrocarbons (PAH), and aromatic and unsaturated olefinic hydrocarbons.

Oil spills, oil transportation, drilling operations, and runoff from refineries and local fuel filling stations are some of the sources of hydrocarbon oil pollution in marine aquatic environments. Hydrocarbon oils are difficult to remove from water because of their oily nature and poor water solubility. The oil pollution persists in nature for long period of time and harms flora and fauna of both terrestrial and aquatic ecosystems.

Bioremediation of hydrocarbon oils in marine aquatic environments typically involves the use of hydrocarbonoclastic microorganisms. Hydrocarbonoclastic microorganisms degrade or “biofractionate” hydrocarbons by utilizing hydrocarbon oils as a food source, “eating” and thereby breaking the hydrocarbon molecules. Hydrocarbonoclastic bacteria (HCB) are an important class of hydrocarbonoclastic microorganisms and are usually present in hydrocarbon contaminated sites. Examples of HCB are disclosed in my U.S. Pat. No. 6,267,888 “Biodispersion as a Method For Removal of Hydrocarbon Oil From Marine Aquatic Environments” (the '888 patent), which patent is fully incorporated by reference herein.

In general, the species or strains of hydrocarbonoclastic bacteria for bioremediation of hydrocarbon oils in marine aquatic environments may be derived from Pseudomonas, Phenylobacterium, Stenotrophomonas, Gluconobacter, Agrobacterium, Vibrio, Acinetobacter, or Micrococcus. Exemplary bacterial strains include Pseudomonas pseudoalkaligenes, Phenylobacterium immobile, Stenotrophomonas maltophilia, Gluconobacter cerinus, or Agrobacterium radiobacter. Yeasts that can also be employed for bioremediation of hydrocarbon oils are also considered hydrocarbonoclastic bacteria herein.

Hydrocarbonoclastic bacteria utilized for bioremediation of hydrocarbon oils in marine aquatic environments may be indigenous to the local environment where the source of the oil pollution is located. Exemplary bacteria are derived from Pseudomonas, Phenylobacterium, Stenotrophomonas, Gluconobacter, Agrobacterium, Vibrio, Acinetobacter, or Micrococcus. Exemplary species include Pseudomonas pseudoalkaligenes, Phenylobacterium immobile, Stenotrophomonas maltophilia, Gluconobacter cerinus, Agrobacterium radiobacter or Pseudomonas alkaligenes.

It is also within the scope of the present disclosure to utilize hydrocarbonoclastic bacteria for bioremediation which have been genetically manipulated or otherwise bioengineered.

The species, strains, or cultures of HCB selected for producing a bioremediation product will be referred to as “active bacteria” herein.

The Exxon Valdez oil spill in 1989 triggered research on developing effective bioremediation products for use on open water oil spills. The US EPA evolved a protocol for the evaluation of bioremediation products before the US Coast Guard and local State agencies use them in open waters. However, bioremediation products that could be used under actual field conditions as an emergency response tool for fuel oil spills were not available. The major focus of the research was targeted towards developing products that acted as polishing tools after the cleanup. Even here success was limited as the time taken for complete remediation was very long and remediation through natural attenuation became more acceptable even though it took much longer time.

In April, 2010 more than 250 million gallons of oil was released in the Gulf of Mexico due to a blowout at the Deepwater Horizon off-shore oil-well that was operated by British Petroleum. This opened up an opportunity and a new challenge for bioremediation products that, although short lived, highlighted the need for production of effective bioremediation products in large quantities at a very short notice.

In the absence of availability of such products, the Deepwater Horizon oil spill was reportedly treated with 1.8 million gallons of the oil dispersant COREXIT™ for faster dispersal of the oil even at the cost of harming local marine life. About 11.4 million gallons of oil was burned and more than 60 million gallons of oily waste water was collected.

Bioremediation of the Deepwater Horizon oil spill would have supplemented the efforts for a safe cleanup of the Deepwater Horizon spill and would have harmed the environment less than did the use of chemical dispersants or the burning of the spilled oil. Even without the Deepwater Horizon oil spill, recent literature indicates that oil spills reported as minor are in fact fairly large but the volume of oil is underreported. This strengthens the importance of bioremediation of oil spills as a more environmentally friendly way to clean up open water oil spills.

The initial goal in responding to oil spills that threaten the coast is to prevent oil from reaching the shore. Emergency responders do not typically consider bioremediation of such oil spills because timely delivery of bioremediation products to the scene is not available. Instead, emergency responders are more likely to use oil absorbent booms or chemical dispersants to get the oil out of the water as quickly as possible.

Bioremediation is relatively safe and promises to be a spill response solution for at least some types of open water oil spills. Effective application techniques and delivery mechanisms are required for the acceptance of bioremediation for treating such oil spills. To be considered as an alternative spill response by first responders, a bioremediation product should ideally meet the following criteria.
It should have a fairly long shelf life, thereby enabling relatively large amounts of bioremediation product to be stored and made available on short notice;
It should be available in a easily spray-able form for ease of application;
It should be available in a ready to use form;
It should not involve any preparatory process at the spill site;
It should be available in large quantities near the target site;
It should be available in easily transportable containers;
It should be easily transported by road, tanker trailer, boat, barge or by air;
It should be manufacturable inside barges, tanker trailers, ships, or the like for ease of storage and transport; and
It should be effective in remediating in a relatively short period of time.

Known bioremediation products do not meet the criteria needed for use at an open water oil spill by emergency responders. A brief survey of such known bioremediation products is given below.

1. Freeze-Dried Bacteria Cultures

Most bioremediation products are two-stage or three-stage systems involving the use of freeze-dried bacteria, a nutrient medium for activating the dried bacteria, and a surfactant for breaking down the hydrocarbon film. Oxygen release compounds may also be provided to supply the oxygen required for growing the bacteria. The freeze-dried bacteria must be kept on dry ice or in a freezer until ready to use. If kept frozen, freeze-dried bacteria are stable for up to one year. During use, nutrients must be added frequently for accelerating the growth of the bacteria. Bioremediation of open water spills is often not considered feasible with freeze-dried bacteria because when nutrients are added to a floating slick, the nutrients disperse in the water and are essentially diluted to background levels.

2. Organic Clay Materials

Clay flakes are engineered to float on seawater and attach to oil. The attached clay flakes then slowly release nutrients to enhance microbial action remediating the oil spill. Although clay flake treatment may offer a way to more effectively apply nutrients in open, nutrient-poor waters, the clay flakes are not expected to impact hydrocarbon toxicity.

3. Bacterial Spores

Powders composed of bacterial spores are effective as a bioremediation product only after hydration with a nutrient solution. The dry bacterial spores have a shelf life of 5 years, but once hydrated, the shelf life is only 3 days.

4. Young Williams, 2008: Patent Application Publication No. 20080138883

Disclosed is a product for bioremediation, including: a) biodegradable carrier and a tablet or powder consisting essentially of microorganisms capable of digesting hydrocarbons, b) an inert material, and c) optionally trace oil in an amount sufficient to maintain the microorganisms in a dormant state, said microorganisms located entirely within said biodegradable carrier, and wherein the biodegradable carrier is directly in contact with the microorganisms. The microorganisms, such as those contained in the family of products, are preferably contained in an inert preparation of inorganic material (e.g., natural clay). Further, a trace amount of oil (e.g., crude oil or oil on which the microorganisms are weaned) is present in the preparation in order to maintain the microorganisms in a dormant state for storage, transport, etc. This preparation may be compressed into tablets that absorb water and hydrocarbons, the tablets dissolve and release the microbes into the contaminant during remediation. The microbes are activated and consume and convert the contaminant into natural byproducts, such as fatty acids, carbon dioxide, water, etc. Once the contaminants have been exhausted, the microbes will either die, return to former natural concentration levels, or be eaten by other organisms. The above disclosure does not indicate a time period for bioremediation of contamination in water and it does not appear to offer a ready to use application.

5. Kumar et al., 2010: Patent Application Publication No. 20100274069:

Disclosed is a bioremediation process for treatment of soils contaminated with aromatic hydrocarbons. A method for bioremediation of highly aromatic hydrocarbon waste, including: a) preparing a microbial blend capable of degrading aromatic hydrocarbons wherein microorganisms in the microbial blend are adsorbed on a biodegradable carrier; b) preparing a biopile comprising a mixture of the hydrocarbon waste, soil and a bulking agent; c) adding the microbial blend into the biopile; d) adding nutrients to promote bioremediation reaction and e) maintaining aeration and moisture of the biopile for a time sufficient to complete the bioremediation of the highly aromatic hydrocarbon waste. The above disclosure offers a five stage process suitable for land application and does not offer a ready to use process suitable for major spills on land or major open water oil spills such as the Deepwater Horizon spill.

6. Daane et al., 2003: U.S. Pat. No. 6,503,746

Disclosed are isolated strains of bacteria that degrade polyaromatic hydrocarbons, a method of isolating the strains of bacteria belonging to Bacillaceae and method of using the strains for bioremediation. The disclosed bacterial strains can be used for bioremediation of polyaromatic hydrocarbon in contaminated environments. The solid substrate is mixed with water and optionally aerated. Other contemplated amendments to the substrate include, but are not limited to various co-substrates, alternate carbon sources, emulsifiers and surfactants. These species of bacteria are not very desirable for bioremediation efforts because of their ability to form endospores during adverse environmental conditions. These endospores can remain dormant for many years and retain the ability to germinate and form vegetative cells, thereby leaving environmental “footprints” even after cleanup is achieved. Hence the strains of bacteria disclosed in Dane, et al. are not considered suitable for oil spill bioremediation.

7. Ganti, 2001: U.S. Pat. No. 6,267,888

My '888 patent discloses the use of indigenous microorganisms for bioremediation of hydrocarbons (including polycyclic aromatic hydrocarbons) in open water oil spills. A two-step process is used for successful bioremediation of polluting oil. Hydrocarbon oil (e.g., diesel oil) is added to an oleophilic fertilizer, and a non-spore forming bacterial consortium is then added to the oil/fertilizer combination. However, long-term storage and subsequent transport of a bioremediation product containing hydrocarbon oils may be undesirable.

8. Mandal et al (2012)

Disclosed is a three stage process for cleanup of contaminated soil and oily sludge. A microbial consortium is immobilized with a suitable carrier material, packed in sterilized polybags (packing size 5-20 kg) and transported to the respective sites for its application on oily waste. The consortium was applied on oily waste by manual spreading at regular intervals of one month. A nutrient formulation was dissolved in water and spread uniformly to the bioremediation site with the help of water sprinkler to enhance the microbial consortium population and to mitigate the effect of initial toxic shock. Mixing of oily sludge and microbes was done by tilling of bioremediation sites. The bioremediation was 95% at the end of 12 months period (which is a long time for an effective spill response product). The product is also not available in a ready to use form and does not meet the other criteria listed above.

Thus there is a need for a method for producing a large quantity of a bioremediation product for bioremediation of an open water oil spill or the like that can:

meet local needs and regulations as well as the needs of emergency responders;

be supplied in ready-to-use form that has a relatively long shelf life (e.g., longer than one year) in such ready-to-use form;

can be manufactured in relatively large quantity lots; and

does not include hydrocarbon oils such as crude oil, diesel oil, kerosene, or the like.

### SUMMARY OF THE DISCLOSURE

Disclosed is a method for producing a large quantity of a bioremediation product, and in particular, a bioremediation product for bioremediating an open water oil spill or the like, that can meet the needs listed above.

In an embodiment, the method includes the step of preparing a seed culture. A seed culture includes the active bacteria that will bioremediate the oil or hydrocarbon pollution. Cultures listed by the US Environmental Protection Agency in the National Product List can be obtained from a culture collection agency such as the American Type Culture Collection (ATCC), a private, nonprofit biological resource center located in Manassas, Va. In other possible embodiments where the bioremediation product is being prepared for possible use in a specific area or locality (for example, for use with oil spills from drilling facilities in a specific area), local bacteria can be obtained for use as a seed culture. Seed cultures have a shelf life of about two years.

The method may further include the step of fermenting the seed culture with a nutrient mixture to obtain a final bioremediation product. The nutrient mixture may be a pre-made nutrient mixture that, in possible embodiments, includes an oleophilic fertilizer having oleic acid, nitrogen (urea), and organic phosphorus. The fertilizer may be mixed and blended with a commercially available vegetable oil, such as Canola oil, or other oleophilic carrier that is not a hydrocarbon oil. The nutrient mixture may have a shelf life of about 3 years.

Fermentation is preferably carried out in a relatively large tank that receives the seed culture, the nutrient mixture, and sufficient water to maintain a desired seed culture to nutrient mixture to water volume ratio. The water can be pre-filtered sea water or fresh water, the water being free from chlorine and pathogens.

Active bacteria typically used to bioremediate hydrocarbon oils are aerobic bacteria, with the bacteria oxidizing the oil. When fermenting aerobic bacteria, the bacteria require an adequate supply of air (oxygen) for growth during fermentation. Aeration can be provided during fermentation by clamp mixers that may continuously mix the tank ingredients or by continuously circulating the tank ingredients through one or more circulating pumps.

After fermentation, the resulting bioremediation product is a ready-to-use liquid containing active bacteria and its own food supply. The liquid is easy to distribute at a pollution site or open water spill site using conventional sprayers.

The bioremediation product is preferably stored in relatively large standardized containers such as 55-gallon drums or Intermediate Bulk Containers (IBCs). IBCs in the United States are available in standard 275 and 330 gallon sizes. Trucks can easily transport 5,000 gallons of bioremediation product in drums or IBCs to a treatment site or to a port for transfer to a ship or barge.

In other possible embodiments, the bioremediation product is stored in a bulk tanker trailer for easy over-the-road transport or is stored in or on ships or barges for over-the-water transport to an open water spill site.

In yet other possible embodiments, the bioremediation product is manufactured and stored on site in tanks, tanker trailers, ships, and/or barges.

The disclosed bulk bioremediation method enables large amounts of bioremediation products to be manufactured and made available for treatment of open water oil spills, enabling emergency responders to respond to a major oil spill with less damage to the environment.

Other objects and features of the disclosure will become apparent as the description proceeds, especially when taken in conjunction with the accompanying drawing sheet.

### BRIEF SUMMARY OF THE DRAWING

FIG. 1 is a flow chart illustrating a method or recipe for producing a bioremediation product.

### DETAILED DESCRIPTION

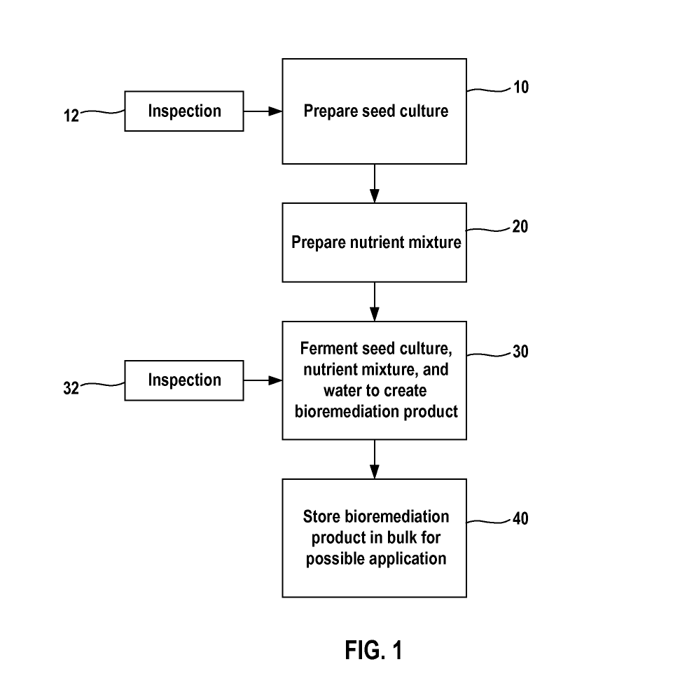

FIG. 1 illustrates a possible embodiment of a recipe or method of producing and storing in bulk a bioremediation product. The illustrated recipe is for production of a bioremediation product usable to bioremediate an open water oil spill such as the Deepwater Horizon oil spill.

The method includes the step 10 of preparing a seed culture that includes the desired active bacteria for bioremediation of hydrocarbon oils. Examples of possible active bacteria that can be used for preparing the seed culture were described above and are disclosed in my '888 patent. The active bacteria can be in water that includes a nutrient mixture (such as the nutrient mixture described in further detail below) to form a nutrient broth that provides the bacteria food for bacterial growth. The seed culture does not include hydrocarbon oil.

In possible embodiments seed cultures are prepared from bacterial cultures listed in the National Contingency Product list maintained by the US Environmental Protection Agency. The cultures can be obtained from ATCC and grown individually in 100 ml of nutrient broth before adding to the seed fermentation tank. In other possible embodiments where the bioremediation product will be stored for possible application in a known area or locality, a local bacteria can be obtained for use as the active bacteria in the seed culture.

When the bacteria preferably reach a concentration of 10̂6 (one US million) per ml, the seed culture is ready for use for bulk production of the bioremediation product. During preparation and storage of the ready-to-use seed culture, regular inspection 12 of the seed culture is conducted to monitor the population of the bacterial consortium and also to monitor any accidental occurrence of pathogens. Shelf life of the ready-to-use seed culture is 2 years.

The method further includes the step 20 of preparing a nutrient mixture. The nutrient mixture will act as a food source for the active bacteria during storage of the bioremediation product.

The nutrient mixture preferably includes an oleophilic fertilizer that includes oleic acid, nitrogen (urea), and organic phosphorus, and a vegetable oil that acts as a carrier. My '888 patent disclosed the use of the oleophilic fertilizer Inipol EAP 22 as a nutrient provider. The illustrated embodiment shown in FIG. 1 replaces the Inipol EAP 22 with a locally manufactured or commercially available oleophilic fertilizer with a composition of nitrogen and phosphorus similar to that of Inipol EAP 22. A preferable commercially available oleophilic fertilizer is S-200™ sold by International Environmental Products, LLC, Villanova, Pa. USA. The vegetable oil is preferably a commercially available vegetable oil like Canola oil or any other suitable medium that is not a hydrocarbon oil. The illustrated nutrient mixture includes a mixture of vegetable oil to fertilizer in the ratio of 1:10 (volume ratio).

The mixture of vegetable oil and oleophilic fertilizer is blended for 60 minutes and the final product is labeled as a nutrient mixture or oleomix and is packed in suitable containers and stored before next use. Shelf life of this nutrient mixture is 3 years.

The method further includes the step 30 of fermenting the seed culture and the nutrient mixture with water to form the bioremediation product. During fermentation, regular inspection 32 of the fermenting mixture is made to monitor the population of the bacterial consortium and also to monitor any accidental occurrence of pathogens.

The illustrated embodiment utilizes a volume ratio of the constituents nutritional mix:water:seed culture of 1:9:0.2, that is, a ratio of 1 gallon of nutritional mixture to 9 gallons of water to 0.2 gallons of seed culture. In an embodiment, 5 gallons of nutritional mix was mixed with 45 gallons of water and 1 gallon of seed culture. Fermentation of the mixture took approximately 5 days (120 hours). Generally, the bioremediation product is considered ready-to-use and ready for storage when the bacteria count in the fermentation tank reaches 10̂6 (one US million) per ml.

The water used in fermentation may be pre-filtered sea water and/or fresh water. The water should be free from chlorine and pathogens. Before setting up production, a water sample is sent for testing of chlorine and pathogens.

Fermentation is preferably carried out in a stainless steel tank of suitable size for the volume of the constituents. Fermentation may be carried out, for non-limiting examples, in stainless steel tanks of 50 gallons, 500 gallons or 1,000 gallons volume. Clamp mixers were used to provide continuous aeration of the fermenting mixture in the tank.

A stainless steel 6000 gallon bulk tanker truck (which includes a tank mounted on a trailer chassis or motor-vehicle chassis configured for on-highway use) has been successfully used to produce 5000 gallons of the bioremediation product in a single batch using the material ratios described above. The fermenting mixture in the tanker tank was continuously circulated by a heavy duty circulating pump.

It is contemplated that fermentation of the bioremediation product could be carried out in even larger tanks on land or on ships or barges with appropriate aeration of the fermenting mixture by recirculation pump or other means.

The method further includes the step 40 of storing the bioremediation product for future use. The remaining nutrients in the bioremediation product nourishes the active bacteria during storage. The stored bioremediation product has a shelf life of about 2 years when stored at normal room temperatures.

In order for the bioremediation product to be effectively employed as a response tool, it is a practical necessity that the bioremediation product be stored in large to very large containers. Standardized storage containers include 55 gallon drums, 300 gallon totes, and Intermediate Bulk Containers (IBCs). These standard containers are easy to transport on trucks and one truck load could deliver 5000 gallons or more of the finished bioremediation product.

These standard containers can also be airlifted for dispersal of bioremediation product directly on an open water oil spill. Oil dispersants were delivered to the Deepwater Horizon oil spill in 300 gallon totes that were released over the floating oil. Bioremediation product can be delivered by air and air-dropped onto open water spills in the same manner.

Bioremediation product can also be stored in bulk tanker trailers that can be towed by fifth-wheel semi tractors. Such bulk tanker trailers are mobile storage units that can be transported anywhere in the US by land in a short period of time and so are ideal for responding to terrestrial oil spills. These “bioremediation on wheels” units can make stops at multiple spill sites. A tanker trailer can also deliver bioremediation product to a port and transfer the product to a boat, ship or a barge to treat an open water oil spill.

Bioremediation products may also be stored in or on ships, boats, or barges to be moved to an open water oil spill with a minimum delay. Boats, ships, or barges could also be located near possible spill sites; for example, boats or barges storing bioremediation product could be moored to an open water oil rig.

Bioremediation products may also be manufactured on boats, ships, barges, and/or tankers if needed while moored at a spill site.

While this disclosure includes one or more illustrative embodiments described in detail, it is understood that the one or more embodiments are each capable of modification and that the scope of this disclosure is not limited to the precise details set forth herein but include such modifications that would be obvious to a person of ordinary skill in the relevant art, as well as such changes and alterations that fall within the purview of the following claims.

What is being claimed is:

1. A method for bulk production of a bioremediation product comprising the steps of:
(a) mixing hydrocarbonoclastic bacteria, oleophilic fertilizer, and an oleophilic carrier that is not a hydrocarbon oil in a container to form a composition; and
(b) fermenting the composition.

2. The method of claim 1 wherein the oleophilic carrier comprises vegetable oil.

3. The method of claim 2 wherein the vegetable oil comprises canola oil.

4. The method of claim 1 wherein the oleophilic fertilizer comprises oleic acid, nitrogen, and phosphorus.

5. The method of claim 1 wherein step (a) further includes the step of:
(c) mixing the bacteria, fertilizer, and carrier with water to form the composition.

6. The method of claim 5 wherein step (a) comprises the step of:
(c) adding bacteria in a form having a bacteria count of 10̂6 per ml.

7. The method of claim 6 wherein the fertilizer and carrier are provided at a volume ratio of fertilizer:carrier of 1:10.

8. The method of claim 6 wherein the fertilizer and carrier are mixed together prior to being mixed with the bacteria.

9. The method of claim 8 wherein the fertilizer and carrier mix is stored for at least two years before performing step (a).

10. The method of claim 8 wherein the bacteria culture, the water, and the fertilizer and carrier mixture are provide at a volume ratio of bacteria culture:water:fertilizer and carrier mixture of 1:5:0.2.

11. The method of claim 1 comprising the step of:
(c) aerating the mixture while performing step (b).

12. The method of claim 11 wherein step (c) comprises continuously circulating the fermenting composition.

13. The method of claim 1 wherein the volume of the fermenting composition is not less than 500 gallons.

14. The method of claim 13 wherein the volume of the fermenting composition is not less than 5,000 gallons.

15. The method of claim 1 further comprising the step of:
(c) after completing step (b), storing the fermented composition in one or more containers for at least two years.

16. The method of claim 1 further comprising the step of:
(c) after completing step (b), applying the fermented composition as a bioremediation product on hydrocarbon oil pollution without further processing.

17. The method of claim 16 wherein step (c) comprises the step of:
(d) air-dropping the fermented composition onto an open water hydrocarbon oil pollution site.

18. The method of claim 1 further comprising the step of:
is located on or forms part of one of the following items (a)-(d): (a) a barge, (b) a boat, (c) a ship, and (d) a bulk tanker truck.

19. The method of claim 1 comprising the steps of:
(c) obtaining hydrocarbonoclastic bacteria having a bacteria count of less than 10̂6 per ml; and
(d) increasing the bacteria count of the obtained bacteria to at least 10̂6 per ml before performing step (a) with the bacteria.

20. The method of claim 1 comprising the step of:
(c) removing the composition from the container when the bacteria count of the bacteria in the fermenting composition is at least 10̂6 per ml.
